(19) United States
(12) Patent Application Publication
(10) Pub. No.: US 2021/0290950 A1
(43) Pub. Date: Sep. 23, 2021

(54) Implantable Tissue Stimulator and Method of Use

(71) Applicant: Micron Medical LLC, Boca Raton, FL (US)

(72) Inventors: Laura Perryman, Boca Raton, FL (US); Benjamin Speck, Boca Raton, FL (US); Graham Patrick Greene, Boca Raton, FL (US)

(21) Appl. No.: 16/822,555

(22) Filed: Mar. 18, 2020

(51) Int. Cl. A61N 1/05; A61N 1/36

(52) U.S. Cl. CPC A61N 1/0558; A61N 1/36062

(57) ABSTRACT

An implantable tissue stimulator assembly includes a stimulator having a housing, a plurality of electrodes positioned along the housing, and a plurality of tines extending outwardly from the housing. An introducer includes a first barrel including a first base and a hollow first cylinder extending outwardly from the first base. The first cylinder is configured to receive the stimulator for insertion of the stimulator into tissue. A first stylet includes a first handle and a first shaft extending outwardly from the first handle, with the first stylet being received in the first cylinder.

### TECHNICAL FIELD

This disclosure relates to an implantable tissue stimulator, and more particularly, an implantable tissue stimulator with tines to prevent migration of the stimulator when implanted in tissue.

### BACKGROUND

Neural modulation of neural tissue in the body by electrical stimulation has become an important type of therapy for chronic disabling conditions, such as chronic pain, problems of movement initiation and control, involuntary movements, dystonia, urinary and fecal incontinence, sexual difficulties, vascular insufficiency, heart arrhythmia and more. Electrical stimulation of the spinal column and nerve bundles leaving the spinal cord was the first approved neural modulation therapy and been used commercially since the 1970s. Implanted electrodes are used to pass pulsatile electrical currents of controllable frequency, pulse width and amplitudes. Two or more electrodes may be in contact with neural elements, typically axons, and can selectively activate varying diameters of axons, with positive therapeutic benefits. A variety of therapeutic intra-body electrical stimulation techniques may be utilized to treat neuropathic conditions that utilize an implanted neural stimulator in the spinal column or surrounding areas, including the dorsal horn, dorsal root ganglia, dorsal roots, dorsal column fibers and peripheral nerve bundles leaving the dorsal column or brain, such as vagus-, occipital-, trigeminal, hypoglossal-, sacral-, unlar-, median, radial-, cluneal, ilioguinal, tibial, and coccygeal nerves.

A stimulator implanted in the body must be secured, or fixated, in the tissue to prevent migration. Fixation of a stimulator into tissue is traditionally done manually by the doctor. The doctor may use sutures and/or various anchoring devices, which are either pierced through the stimulator or affixed to the stimulator during the implantation procedure.

### SUMMARY

In general, this disclosure relates to an improved tissue stimulator to be positioned proximate a target location within tissue and an introducer for use in inserting the tissue stimulator into the tissue.

In accordance with one aspect, an implantable tissue stimulator assembly includes a stimulator having a housing, a plurality of electrodes positioned along the housing, and a plurality of tines extending outwardly from the housing. An introducer includes a first barrel including a first base and a hollow first cylinder extending outwardly from the first base. The first cylinder is configured to receive the stimulator for insertion of the stimulator into tissue. A first stylet includes a first handle and a first shaft extending outwardly from the first handle, with the first stylet being received in the first cylinder.

In accordance with another aspect, an implantable tissue stimulator assembly includes a stimulator having a housing and a plurality of electrodes proximate a distal end of the housing. A plurality of first tines extend outwardly from the housing proximate the distal end, and a second plurality of tines extend outwardly from the housing proximate a proximal end of the housing. A first introducer includes a first barrel including a first base and a hollow first cylinder extending outwardly from the base. The first base and first cylinder include a pair of opposed perforations extending along a length of the first base and the first cylinder. The first cylinder is configured to receive the distal end of the stimulator for insertion of the stimulator into tissue, and the first tines are configured to be folded inwardly toward the housing when the stimulator is received in the first cylinder. A first stylet includes a first handle and a first shaft extending outwardly from the first handle, the first stylet being received in the first cylinder. A second introducer is configured to receive the proximal end of the stimulator for insertion of the stimulator into tissue. The second introducer includes a second barrel including a second base and a hollow second cylinder extending outwardly from the second base, with the second base and second cylinder including a pair of opposed perforations extending along a length of the second base and the second cylinder. A second stylet includes a second handle and a second shaft extending outwardly from the second handle, with the second stylet being received in the second cylinder.

In accordance with a further aspect, a method of implanting a tissue stimulator includes the steps of inserting a first introducer through an insertion site of tissue such that a tip of the introducer is proximate a target site within the tissue, the introducer including a first barrel including a first base and a hollow first cylinder extending outwardly from the first base, and a first stylet including a first handle and a first shaft extending outwardly from the first handle, the first stylet being received in the first cylinder; removing the first stylet from the first cylinder; inserting a distal end of a stimulator into the first cylinder, the stimulator having a housing, a plurality of electrodes connected to the housing proximate a distal end of the housing, and a plurality of first tines extending outwardly from the housing proximate the plurality of electrodes, the first tines being folded inwardly toward the housing when the stimulator is in the first cylinder; and removing the first barrel from the tissue such that the first tines move outwardly away from the housing and engage the tissue.

Additional aspects, configurations, embodiments and examples are described in more detail below.

### DESCRIPTION OF DRAWINGS

Certain manufacturing techniques and manufactured devices are described below with reference to the accompanying figures.

FIG. 1 is an elevation view of an implantable tissue stimulator.

FIG. 2 is an elevation view of a first introducer for use in implanting the tissue stimulator of FIG. 1

FIG. 3 is an exploded view of the first introducer of FIG. 2.

FIG. 4 is an exploded view of an alternative embodiment of the first introducer of FIG. 2.

FIG. 5 is a schematic illustration of the first introducer of FIG. 2 being inserted into a target location within tissue.

FIG. 6 is a schematic illustration of the first introducer of FIG. 2 and the tissue stimulator of FIG. 1, shown after the stylet of the first introducer has been removed from the tissue and before the tissue stimulator has been inserted into the barrel of the first introducer.

FIG. 7 is a schematic illustration of the tissue stimulator of FIG. 1 inserted into the barrel of the first introducer of FIG. 2.

FIG. 8 is a schematic illustration showing the barrel of the first introducer of FIG. 2 being removed from the tissue.

FIG. 9 is a schematic illustration showing the barrel of the first introducer of FIG. 2 being removed from the tissue as the barrel is being split into two portions.

FIG. 10 is a schematic illustration showing the distal end of the stimulator in position in the tissue, and the barrel of the first introducer of FIG. 2 in two separate portions after being removed from the tissue.

FIG. 11 is a schematic illustration showing a second introducer being inserted into the tissue.

FIG. 12 is a schematic illustration showing the barrel of the second introducer of FIG. 11 in position in the tissue after the stylet of the second introducer has been removed.

FIG. 13 is a schematic illustration of posterior end of the tissue stimulator of FIG. 1 prior to being inserted into the barrel of the second introducer of FIG. 11.

FIG. 14 is a schematic illustration showing the posterior end of the tissue stimulator in position in the tissue and showing the barrel of the introducer of FIG. 11 being removed from the tissue as the barrel is being split into two portions.

FIG. 15 is a schematic illustration showing the complete tissue stimulator in position in the tissue, and the barrel of the second introducer of FIG. 11 in two separate portions after being removed from the tissue.

FIG. 16 is a perspective view of an alternative embodiment of the first introducer of FIG. 2.

FIG. 17 is a section view of an alternative embodiment of the barrel of the first introducer of FIG. 2, shown with the tissue stimulator being inserted into the barrel.

FIG. 18 is a section view of the barrel of FIG. 17, shown with the tines of the tissue stimulator partially folded toward the housing of the tissue stimulator.

FIG. 19 is a perspective view, partially broken away, of the tissue stimulator of FIG. 1, showing sleeves and tines of the tissue stimulator.

FIG. 20 is an elevation view, partially broken away, of an alternative embodiment of the tissue stimulator of FIG. 1.

FIG. 21 is a plan view, partially broken away, of an alternative embodiment of a tine of the tissue stimulator of FIG. 1.

FIG. 22 is a plan view, partially broken away, of another alternative embodiment of a tine of the tissue stimulator of FIG. 1.

FIG. 23 is a section view of a tine of the tissue stimulator of FIG. 1.

FIG. 24 is a section view of an alternative embodiment of a tine of the tissue stimulator of FIG. 1, shown with ribs on the tine.

FIG. 25 is a section view of an alternative embodiment of a tine of the tissue stimulator of FIG. 1, shown with a lubricous material on an exterior of the tine.

FIG. 26 is a rear perspective view of an alternative embodiment of a sleeve and tines of the tissue stimulator of FIG. 1.

FIG. 27 is a front perspective view of the sleeve and tines of FIG. 26.

FIG. 28 is a schematic view of another alternative embodiment of the sleeve and tines of the tissue stimulator of FIG. 1.

FIG. 29 is an elevation view of the tissue stimulator of FIG. 1 shown with the sleeve and tines of FIG. 26.

FIG. 30 is an elevation view, partially in section, showing the tissue stimulator of FIG. 29 being inserted into a first barrel.

FIG. 31 is an elevation view, partially in section, showing the tissue stimulator of FIG. 29 being inserted into a first barrel, with the tines being folded inwardly toward the housing of the tissue stimulator.

FIG. 32 is an end elevation view of an alternative embodiment of the sleeve of the tissue stimulator of FIG. 1.

FIG. 33 is an elevation view, partially in section, showing a tine secured to the sleeve of FIG. 32.

FIG. 34 is an elevation view, partially in section, showing an alternative embodiment of a tine secured to the sleeve of FIG. 32.

FIG. 35 is an elevation view of an alternative embodiment of the tissue stimulator of FIG. 1, shown with tines extending perpendicular to the housing of the tissue stimulator.

FIG. 36 is an elevation view of the tissue stimulator of FIG. 1, shown with an alternative embodiment of the tines.

FIG. 37 is an elevation view of the tissue stimulator of FIG. 1, shown with another alternative embodiment of the tines.

FIG. 38 is an elevation view of the tissue stimulator of FIG. 1, shown with yet another alternative embodiment of the tines.

FIG. 39 is an elevation view of the tissue stimulator of FIG. 1, shown with a further alternative embodiment of the tines.

FIG. 40 is a perspective view of an alternative embodiment of the sleeve and tines of the tissue stimulator of FIG. 1.

FIG. 41 is a plan view of the sleeve and tines of FIG. 40.

The figures referred to above are not drawn necessarily to scale and should be understood to provide a representation of the invention, illustrative of the principles involved. Some features of the implantable tissue stimulator depicted in the drawings have been enlarged or distorted relative to others to facilitate explanation and understanding. The same reference numbers are used in the drawings for similar or identical components and features shown in various alternative embodiments. Implantable tissue stimulators as disclosed herein would have configurations and components determined, in part, by the intended application and environment in which they are used.

### DETAILED DESCRIPTION

Certain improvements to processes for applying an insulator to an implantable tissue stimulator are described. The various processes may include injection molding (e.g., using over molding or insert molding or a combination thereof) and/or heat treatment of a reflowable insulating material.

The term “approximately” as used herein is meant to mean close to, or about a particular value, within the constraints of sensible commercial engineering objectives, costs, manufacturing tolerances, and capabilities in the field of plyometric box manufacturing and use. Similarly, the term “substantially” as used herein is meant to mean mostly, or almost the same as, within the constraints of sensible commercial engineering objectives, costs, manufacturing tolerances, and capabilities in the field of plyometric box manufacturing and use.

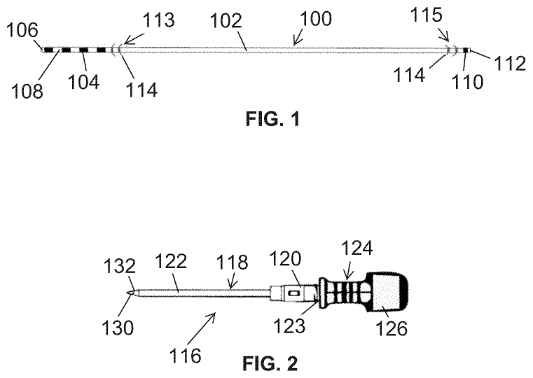

FIG. 1 shows an example of an implantable tissue stimulator 100 configured to be implanted within a patient's body for delivering electrical therapy to tissues within the body. Tissue stimulator 100 may have an exterior design that provides strength and a smooth profile for optimal insertion and performance within the patient. For example, tissue stimulator 100 may include a housing 102, which may be molded (e.g., overmolded or insert molded) of a clear material around various internal components of tissue stimulator 100. Accordingly, tissue stimulator 100 may be referred to as a monolithic device for which electronic components are secured to one small, flat substrate that may be delivered to the body through an introducer, described in greater detail below.

Tissue stimulator 100 may include a circuit board and various circuit components (not shown here), and electrodes 104 that are connected to the circuit board and positioned proximate a distal end 106 of housing 102, and separated from one another by spacers 108. An antenna 110 may also be connected to the circuit board and may be positioned in housing 102, proximate a proximal end 112 of housing 102.

Stimulator 100 may include a plurality of fixation elements or tines 114 that extend outwardly from housing 102. Tines 114 may serve to anchor stimulator 100 in tissue in a body, as described in greater detail below, so as to prevent migration of stimulator 100 after it has been implanted in the body.

In the illustrated embodiment, a first set 113 of tines 114 are positioned on housing 102 proximate distal end 106, with electrodes 104 being positioned between first set 113 and distal end 106. A second set 115 of tines 114 may be positioned on housing 102 proximate proximal end 112, with antenna 110 being positioned between second set 115 and proximal end 112.

It is to be appreciated that tines 114 can be positioned at various locations along housing 102. It is to be appreciated that in certain embodiments, tines 114 may be positioned only proximate distal end 106. In other embodiments, tines 114 may be positioned at additional locations other than proximate distal end 106 and proximal end 112 as illustrated here. Other suitable locations for tines 114 will become readily apparent to those skilled in the art, given the benefit of this disclosure.

Introducers may be used to assist in implanting stimulator 100 in tissue in the body in a minimally invasive manner through a single incision in the tissue. An embodiment of a first introducer 116 is seen in FIGS. 2-3. First introducer 116 may include a first barrel 118. First barrel 118 may include a first base 120 and a hollow first cylinder 122 extending outwardly from first base 120. First cylinder 122 may be configured to receive stimulator 100 in an open end 123 of first base 120 for insertion of stimulator 100 into tissue in the body, as described in greater detail below.

First barrel 118 may be formed of a plastic material, such as a resin. Exemplary materials for first barrel 118 include hytrel, polytetrafluroethylene (PTFE), and high density polyethylene (HDPE). Other suitable materials for first barrel 118 will become readily apparent to those skilled in the art, given the benefit of this disclosure.

First introducer 116 may also include a first stylet 124 that may include a first handle 126 and a first shaft 128 extending outwardly from first handle 126. First shaft 128 may be received in first cylinder 122 such that a tip 130 of first shaft 128 projects out of a distal end 132 of first cylinder 122. Tip 130 may be pointed so as to pierce tissue as introducer 116 is inserted into tissue in a body. In other embodiments, tip 130 may be blunt, which may be beneficial when implanting tissue stimulator in the vicinity of a nerve or blood vessel.

First handle 126 may be configured so as to provide a doctor or any other medical personnel with a convenient and comfortable grip to help insert introducer 116 into tissue in the body. A portion of a distal end 134 of first handle 126 may be received in first base 120.

It is to be appreciated that the length of introducers can be varied to facilitate inserting stimulator 100 to different depths within tissue of the body. As illustrated in FIG. 2, first introducer 116 may have a total length L1 of between approximately 3 inches and approximately 10 inches, and more particularly, approximately 4.5 inches. In such an embodiment first barrel 118 may have a total length L2 of between approximately 3 inches and approximately 10 inches.

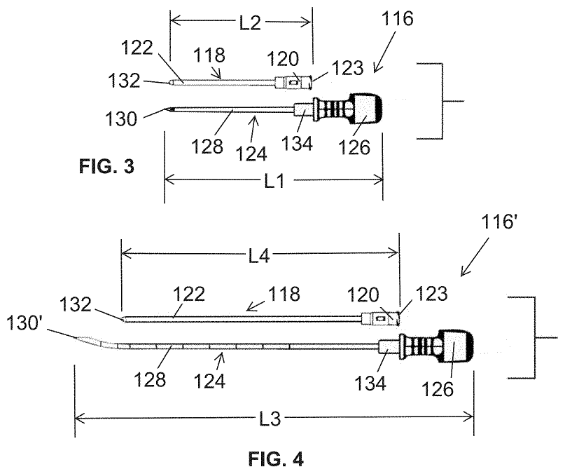

In other embodiments, as seen in FIG. 4, a first introducer 116′ may have a total length L3 that is longer than length L1 to allow stimulator 100 to be inserted to a deeper depth within tissue. As illustrated here, first introducer 116′ may have a total length L3 of between approximately 4 inches and approximately 10 inches, and more particularly, approximately 6 inches. In such an embodiment, first barrel 118 may have a total length L4 of between approximately 3 inches and approximately 10 inches, and more particularly, approximately 5.5 inches.

It is to be appreciated that the lengths of first introducer 116 and its components is not restricted to the lengths listed above, and that other suitable lengths for first introducer 116 and its components will become readily apparent to those skilled in the art, given the benefit of this disclosure.

In certain embodiments, as illustrated in FIG. 4, tip 130′ on first shaft 128 may be curved to facilitate inserting stimulator 100 into tissue along a curved, or non-linear track within tissue of the body.

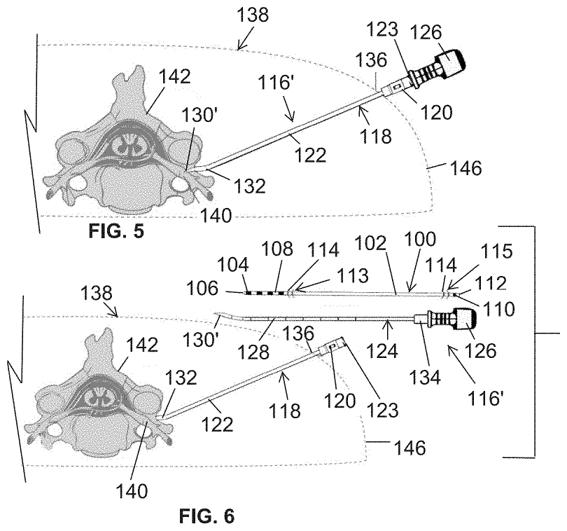

A method of inserting stimulator 100 into an incision 136 in tissue 138 is described as follows in conjunction with FIGS. 5-13. In the embodiment illustrated herein, stimulator 100 may be implanted in order to target a dorsal root ganglion (“DRG”) 140 proximate a spine 142.

In this embodiment first introducer 116′ with a bent tip 130′ on first shaft 128 of first stylet 124 may be used to reach DRG 140. Since tip 130′ extends beyond distal end 132 of first barrel 118, tip 130′ pierces tissue 138 to create a path or tunnel through which the remainder of first introducer 116′ follows. First introducer 116′ may be inserted through incision 136 into tissue 138 until tip 130′ and distal end 132 of first barrel 118 are proximate DRG 140, as illustrated in FIG. 5. Once it has been determined that tip 130′ and distal end 132 are in the correct position proximate DRG 140, first barrel 118 may be held in a stationary position while first stylet 124 is withdrawn from first barrel 118. As illustrated in FIG. 6, first stylet 124 is shown removed from first barrel 118, first barrel 118 is shown in the same position within tissue 138 such that distal end 132 is proximate DRG 140. Stimulator 100 is seen here prior to insertion into first barrel 118.

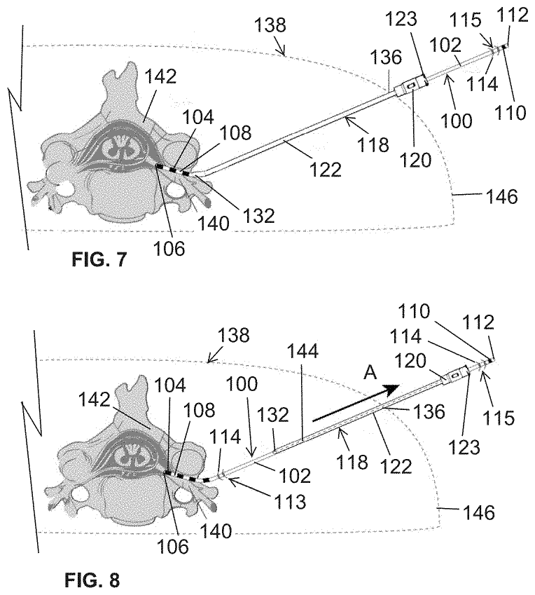

As illustrated in FIG. 7, distal end 106 of stimulator 100 may be inserted into open end 123 of first barrel 118, and moved onward through first cylinder 122 until distal end 106 and electrodes 104 of stimulator 100 are in position proximate the target DRG 140. As stimulator 100 moves inwardly through first cylinder 122 of first barrel 118, tines 114 of first set 113 (not visible) are folded inwardly toward housing 102, and first cylinder 122 prevents tines 114 from engaging tissue 138. When distal end 106 and electrodes 104 are in the proper position proximate target DRG 140, second set 115 of tines 114 are positioned outward of first barrel 118.

After distal end 106 and electrodes 104 are placed in the proper position proximate target DRG 140, first barrel 118 may be retracted from tissue 138 while stimulator 100 is held in place with respect to tissue 138 by the user. As first barrel 118 is retracted, the portion of stimulator 100 that had been encased within first cylinder 122 is exposed, and tines 114 of first set 113 move outwardly away from housing 102 to their original position and engage tissue 138, thereby fixing stimulator 100 with respect to tissue 138.

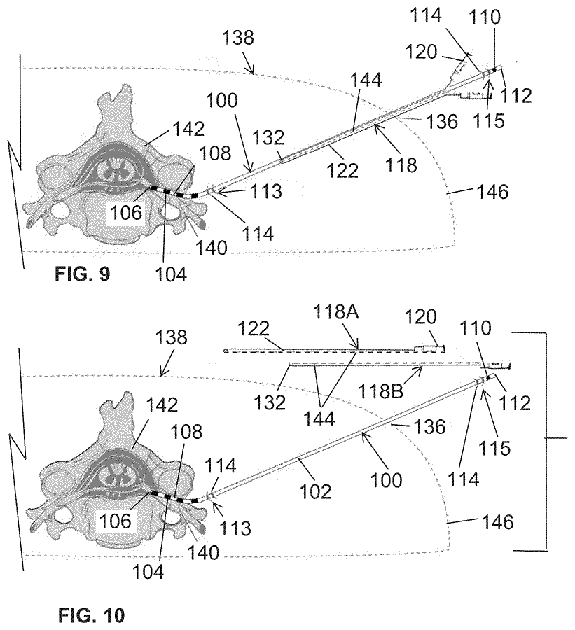

In certain embodiments, as illustrated in FIGS. 8-10, first barrel 118 may have a break-away feature that facilitates implantation of stimulator 100 in tissue 138. As illustrated here, first barrel 118 may have a pair of opposed grooves, score lines, or perforations 144 (only one visible in FIG. 8) extending along a length of first base 120 and first cylinder 122 of first barrel 118. As first barrel 118 is begun to be removed from tissue 138 through incision 136, opposed sides of first base 120 of first barrel 118 may be grasped by the user and pulled apart from one another. As first barrel 118 is pulled out of tissue 138, first barrel 118 may be torn or split in a controlled fashion along perforations 144 into two portions 118A and 118B, as illustrated in FIG. 9. By splitting first barrel 118 into two portions as it is removed from tissue 138 through incision 136, the user may avoid impinging upon and disturbing second set 115 of tines 114.

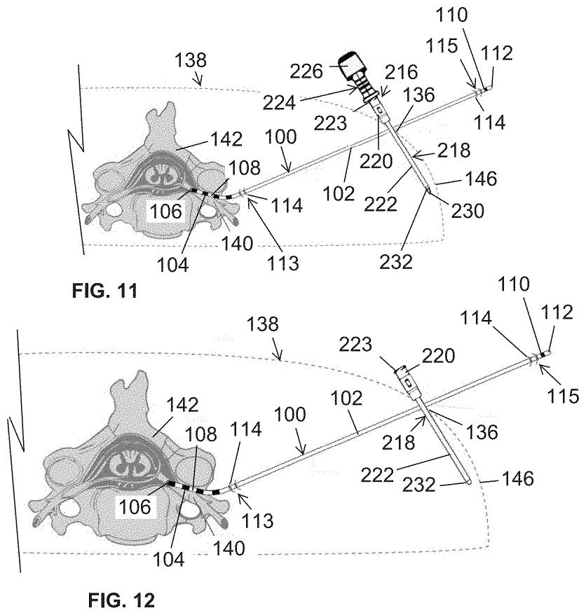

In certain embodiments, as illustrated in FIGS. 11-15, proximal end 112 of stimulator 100 may also be implanted within tissue 138. The implantation of proximal end 112 is accomplished in a similar manner to that of the implantation of distal end 106. However, rather than implanting proximal end 106 proximate target DRG 140, proximal end 106 is implanted proximate surface 146 of tissue 138, thereby enhancing the reception of antenna 110 and operation of stimulator 100.

As seen in FIG. 11, a second introducer 216 may be used for the insertion of proximal end 112 of stimulator 100 into tissue 138. As shown here, second introducer 216 is inserted through incision 136 into tissue 138 in a direction chosen such that tip 230 and distal end 232 are positioned just beneath and proximate surface 146 of tissue 138. As described above with respect to first introducer 116, tip 230 pierces tissue 138 and creates a path or tunnel through which second cylinder 222 of second barrel 218 can travel.

When tip 230 and distal end 232 of second cylinder 222 reach the desired position within tissue 138 proximate surface 146, second barrel 218 may be held stationary while second stylet 224 is removed from second barrel 218.

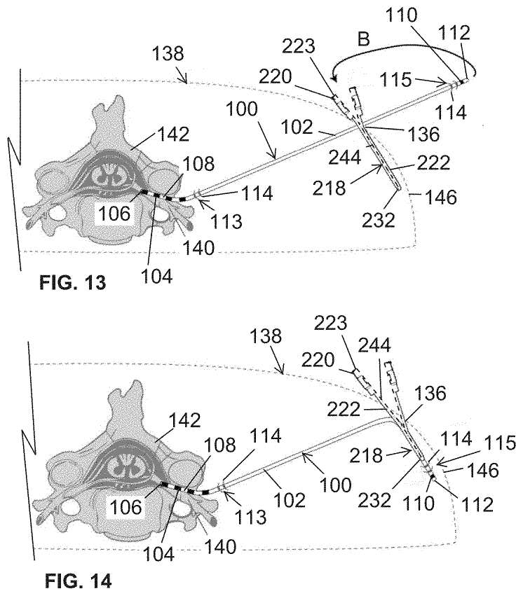

As illustrated in FIG. 13 by arrow B, proximal end 112 of stimulator 100 may be inserted into open end 223 of second barrel 218, and moved onward through second cylinder 222 until proximal end 112 of stimulator 100 is in position proximate surface 146 of tissue 138. As stimulator 100 moves inwardly through second cylinder 222 of second barrel 218, tines 114 of second set 115 (not shown) are folded inwardly toward housing 102, and second cylinder 222 prevents tines 114 of second set 115 from engaging tissue 138.

After proximal end 112 is placed in the proper position proximate surface 146 of tissue 138, second barrel 218 may be retracted from tissue 138 while stimulator 100 is held in place with respect to tissue 138 by the user. As second barrel 218 is retracted, the portion of stimulator 100 that had been encased within second cylinder 222 is exposed, and tines 114 of second set 115 move outwardly away from housing 102 to their original position and engage tissue 138, thereby fixing stimulator 100 with respect to tissue 138.

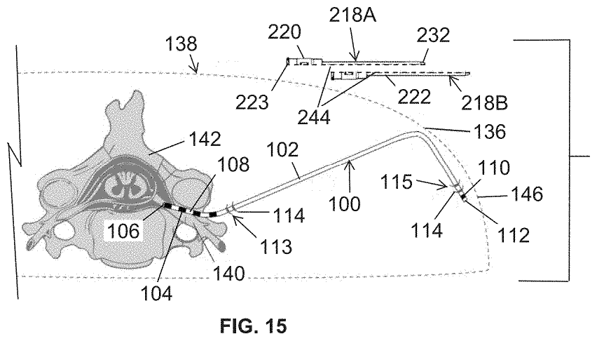

In certain embodiments, as illustrated in FIGS. 13-15, second barrel 218 may have a break-away feature that facilitates implantation of stimulator 100 in tissue 138. As illustrated here, second base 220 and second cylinder 220 of second barrel 218 may have a pair of opposed grooves, score lines, or perforations 244 (only one visible in FIGS. 13-14) extending along a length of second base 220 and second cylinder 220. As second barrel 218 is begun to be removed from tissue 138 through incision 136, opposed sides of second base 220 of second barrel 218 may be grasped by the user and pulled apart from one another. As second barrel 218 is pulled out of tissue 138, second barrel 218 may be torn or split in a controlled fashion along perforations 244 into two portions 218A and 218B, as illustrated in FIG. 15.

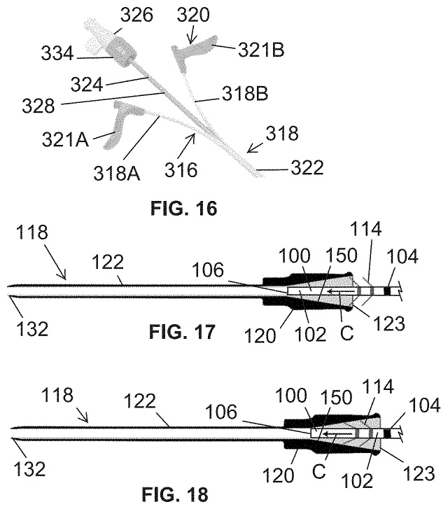

Another embodiment of a first introducer first introducer 316 is seen in FIG. 16. As seen here, first stylet 324 may include a first handle 326. First shaft 328 may extend outwardly from distal end 324 of first handle 326. First barrel 318 with first cylinder 322 may be perforated so it can be split into two portions 318A and 318B as it is removed from tissue 138 to avoid impinging upon and disturbing tines 114. To facilitate splitting first barrel 318, first base 320 of first barrel 318 may include a pair of handles 321A and 321B, with handle 321A being connected to portion 318A of first barrel 318 and handle 321B being connected to portion 318B of first barrel 318. To split barrel 318, a user may grasp handles 321A and 321B and pull them apart, thereby facilitating the splitting of first barrel 318 into portions 318A and 318B. It is to be appreciated that second introducer 216 could also have a configuration as illustrated here with handles on its second base 220 to facilitate splitting second barrel 218.

In certain embodiments, as illustrated in FIGS. 17-18, open end 123 of first base 120 of first barrel 118 may have an inwardly tapered sidewall 150 creating a funnel into which distal end 106 of stimulator 100 may be inserted. As distal end 106 of stimulator 100 is moved forwardly into first base 120 through open end 123 in the direction of arrow C, tapered sidewall 150 facilitates the folding of tines 114 toward housing 102 of stimulator 100. This controlled folding of tines 114 inwardly toward housing 102 protects tines 114 and helps prevent them from being bent backwards and damaged as stimulator 100 is moved forwardly through cylinder 122 of first barrel 118.

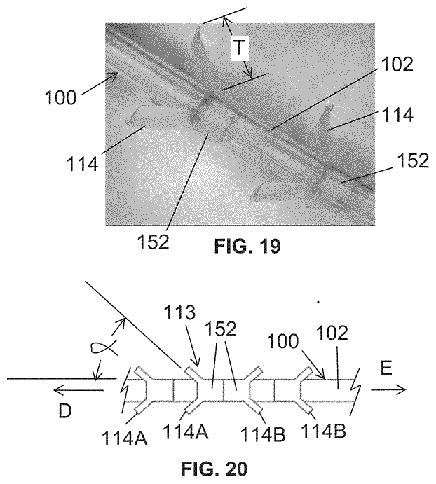

In certain embodiments, as illustrated in FIG. 19, stimulator 100 may include one or more sleeves 152 that surround housing 102 of stimulator 100, with a plurality of tines 114 extending outwardly from each sleeve 152. Sleeves 152 may be slid onto housing 102 of stimulator 100 from distal end 106 or proximal end 112. In certain embodiments, tines 114 and sleeve 152 may be formed as a unitary, one-piece element of monolithic construction, such as by injection molding, for example. In other embodiments, tines 114 and sleeve 152 may be formed as separate elements and then joined together by welding or other suitable fastening means.

In the illustrated embodiment, three separate tines 114 extend outwardly from each sleeve 152. It is to be appreciated that more or less than three individual tines 114 may extend outwardly from each sleeve 152.

In certain embodiments, tines 114 may have length T between approximately 1.5 mm and approximately 5 mm, and more particularly approximately 2 mm. The selected length for tines 114 may be determined based on the type of tissue in which tissue stimulator 100 is to be implanted. It is to be appreciated that all tines 114 of a single tissue stimulator 100 do not necessarily need to be the same length. Thus for example some tines 114, such as those in first set 113, for example, could have a first length with those in second set 115 having a second length that is different than the first length.

In certain embodiments, as illustrated in FIG. 20, tines 114 may be oriented to point in different directions. For example, as seen here, first set 113 of tines 114 may include tines 114A that are angled forwardly in the direction of arrow D, and tines 114B that are angled in the opposite direction, or rearwardly in the direction of arrow E. Thus, after stimulator 100 is inserted through first barrel 118 into is desired position within tissue 138, and first barrel 118 is removed from tissue 138, forwardly extending tines 114A prevent migration of stimulator 100 forwardly within tissue 138, and rearwardly extending tines 114B prevent migration of stimulator 100 rearwardly within tissue 138.

As seen in FIG. 20, tines 114 may extend upwardly and outwardly at an angle α with respect to housing 102 of stimulator 100. In certain embodiments, angle α may be between approximately 30 degrees and approximately 90 degrees, and more particularly approximately 45 degrees. It is to be appreciated that all tines 114 of a single tissue stimulator 100 do not necessarily need to be oriented at the same angle. Thus for example some tines 114, such as those in first set 113, for example, could be positioned at a first angle with those in second set 115 being positioned at a second angle that is different than the first angle.

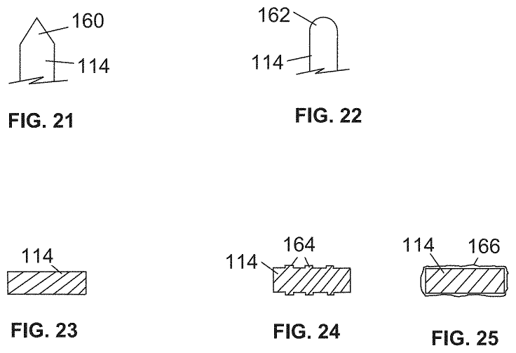

It is to be appreciated that tines 114 may have different shapes, profiles, lengths, and cross-sections, and may have varied material properties. For example, as illustrated in FIG. 21, one or more tines 114 may have a pointed tip 160. In other embodiments, as illustrated in FIG. 22, one or more tines 114 may have a rounded tip 162. The configuration and shape of the tip of tines 114 may be varied depending on the type of tissue that tines 114 will be embedded in after insertion. Other suitable shapes for the tip of tines 114 will become readily apparent to those skilled in the art, given the benefit of this disclosure.

As illustrated in FIG. 23, one or more tines 114 may have a rectangular cross-section. It is to be appreciated that tines 114 could have other cross-sectional shapes, such as a racetrack shape, for example. Other suitable cross-sectional shapes for tines 114 will become readily apparent to those skilled in the art, given the benefit of this disclosure.

In certain embodiments, as illustrated in FIG. 24, a plurality of ribs 164 may be located on exterior surfaces of one or more of tines 114. Ribs 164 may serve to provide enhanced rigidity or stiffness for tines 114. As illustrated here, tine 114 has a rectangular cross-section with three ribs 164 positioned on each of opposed longer sides of tine 114. It is to be appreciated that any number of ribs 164 may be positioned on tine 114, and that ribs 164 may be positioned at any location along the exterior surface of tine 114.

Tines 114 may also be formed of different materials in order to vary the rigidity or stiffness of tines 114. The material stiffness, which may be measured by the Young's modulus of the material, can be chosen based on the types of tissues in which tissue stimulator 100 is to be implanted. Suitable materials for tines 114 include silicone, polyurethane, and HDPE. Other suitable materials for tines 114 will become readily apparent to those skilled in the art, given the benefit of this disclosure.

In certain embodiments, a lubricous material 166 may be positioned on an exterior surface of one or more tines 114 to decrease the frictional force as tines 114 pass through tissue 138. Exemplary lubricous materials include silicones, perfluoropolyether (PFPE), and parylene. Other suitable lubricous materials will become readily apparent to those skilled in the art, given the benefit of this disclosure.

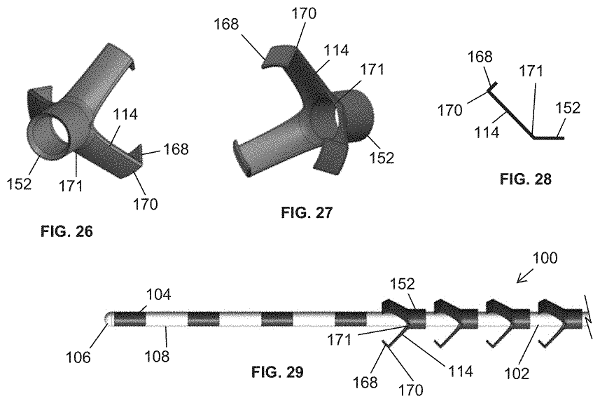

Another embodiment of tines 114 is illustrated in FIGS. 26-29. In this embodiment, a finger or projection 168 is positioned at a distal end 170 of each tine 114, with a proximal end 171 of tine 114 being connected to sleeve 152. Projections 168 may extend inwardly from distal end 170 toward housing 102 of tissue stimulator 100, as seen in FIG. 29. In certain embodiments, projections 168 may extend substantially perpendicular to tines 114. It is to be appreciated that projections 168 may extend inwardly at angles other than perpendicular to tines 114 including acute and obtuse angles with respect to tines 114. Projections 168 may help to engage tissue 138 when tissue stimulator 100 and tines 114 are in the targeted position within tissue 138, thereby helping prevent migration of tissue stimulator 100.

In another embodiment, as illustrated in FIG. 28, projections 168 may extend outwardly away from tines 114 and housing 102 of tissue stimulator 100. In such an embodiment, projections 168 may extend substantially perpendicular to tines 114. It is to be appreciated that projections 168 may extend outwardly at angles other than perpendicular to tines 114 including acute and obtuse angles with respect to tines 114.

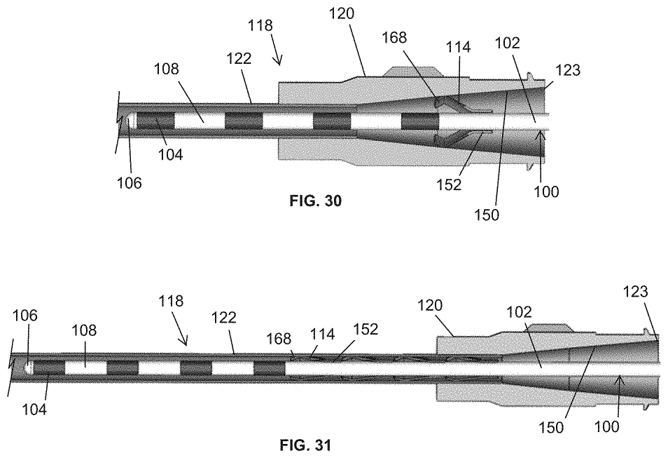

An embodiment showing four sets of tines 114 with projections 168 is illustrated in FIG. 29, where it can be seen that projections 168 extend inwardly toward housing 102 of tissue stimulator 100. FIGS. 30-31 illustrate the introduction of tissue stimulator 100, with tines 114 including projections 168, into first barrel 118, and the folding inwardly of tines 114 and projections 168 as they move forwardly along tapered sidewall 150. As seen in FIG. 31, when tines 114 are folded inwardly toward housing 102 to their greatest extent, projections 168 may flex forwardly so that they are no longer substantially perpendicular to tines 114. After first barrel 118 is removed from tissue 138, as described above, tines 114 and projections 168 may spring back outwardly away from housing 102 of tissue stimulator 100 and engage tissue 138, thereby helping prevent migration of tissue stimulator 100.

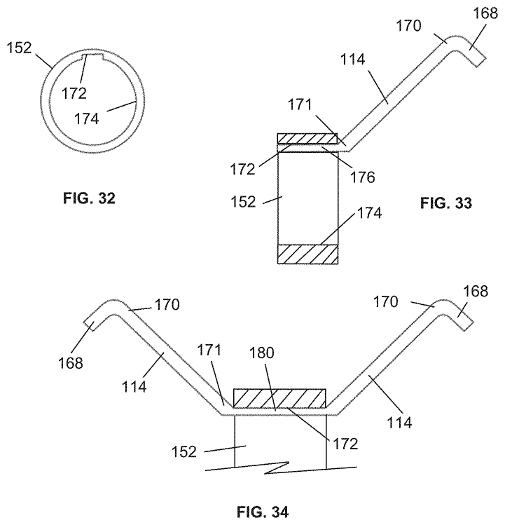

Another embodiment of sleeve 152 is illustrated in FIGS. 32-33. In this embodiment, sleeve 152 and tines 114 are formed as separate elements, such as by injection molding, extrusion, or thermoforming, for example, and are then secured to one another. In this embodiment, a channel 172 may be formed on an interior surface 174 of sleeve 152. A tine base 176 may be positioned at proximal end 171 of tine 114, and tine base 176 may be seated in channel 172. Tine base 176 and sleeve 152 may then be secured or fastened to one another by crimping, swaging, gluing, solvent bonding, or welding, for example. In other embodiments, tine base 176 and sleeve 152 may be secured to one another by a mechanical interlock, such as a snap-fit fastener or any other suitable mechanical interlock. Other suitable methods of securing tine base 176 to sleeve 152 will become readily apparent to those skilled in the art, given the benefit of this disclosure.

Another embodiment is illustrated in which two tines 114 may connected to one another by a common tine base 180, with common tine base 180 being seated in channel 172 of sleeve 152 such that one of the tines 114 extends outwardly from common tine base 180 in a first direction while the other tine 114 extends outwardly from common tine base 180 in an opposed second direction.

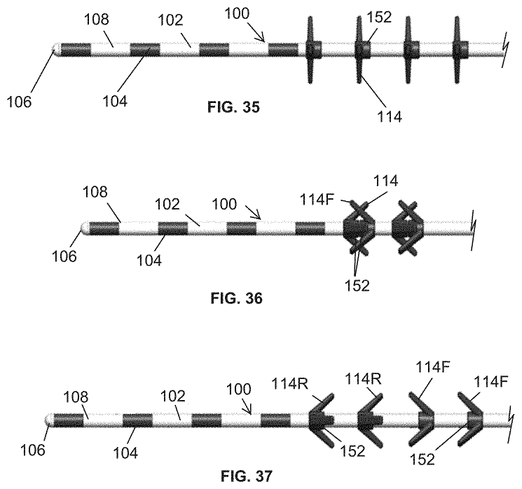

As noted above, tines 114 can be oriented in different manners with respect to housing 102 of tissue stimulator 100. In certain embodiments, as illustrated in FIG. 35, tissue stimulator 100 may include a plurality of tines 114 that extend outwardly in a substantially perpendicular direction from sleeve 152 and housing 102 of tissue stimulator 100.

In another embodiment, as illustrated in FIG. 36, pairs of sleeves 152 with tines 114F, 114R may be seated next to one another in abutting fashion such that the adjacent sleeves 152 of are in contact with one another. In such an embodiment, tines 114F on one sleeve 152 may be oriented such that they project in a forward direction toward distal end 106 of housing 102 of tissue stimulator 102, while tines 114R on the adjacent sleeve 152 may be oriented so as to project in a rearward direction toward posterior end 112 (not visible here) of housing 102 of tissue stimulator 102. Having tines 114F and 114R project in different directions may help prevent migration of tissue stimulator 100 within tissue 138.

In yet another embodiment, as illustrated in FIG. 37, a first pair of sleeves 152 with tines 114R may be positioned forwardly of a second pair of sleeves 152 with tines 114F such that tines 114R project in a rearward direction toward posterior end 112 (not visible here) of housing 102 of tissue stimulator 102, and tines 114F project in a forward direction toward distal end 106 of housing 102 of tissue stimulator 102. Having tines 114F and 114R project in different directions may help prevent migration of tissue stimulator 100 within tissue 138.

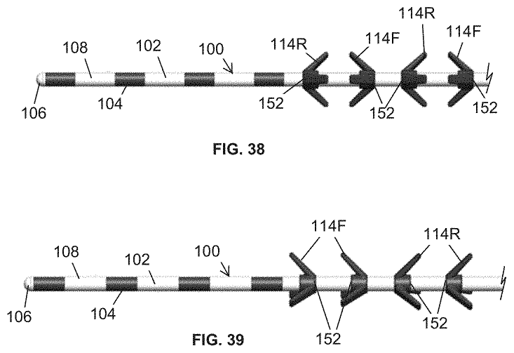

In a further embodiment, as illustrated in FIG. 38, a first pair of sleeves 152 may be positioned forwardly of a second pair of sleeves 152, with a forwardmost sleeve 152 of each pair including rearwardly facing tines 114R and a rearwardmost sleeve 152 of each pair including forwardly facing tines 114F. Having tines 114F and 114R project in different directions may help prevent migration of tissue stimulator 100 within tissue 138.

In another alternative embodiment, as illustrated in FIG. 39, a first pair of sleeves 152 may be positioned forwardly of a second pair of sleeves 152, with the forwardmost pair of sleeves 152 including forwardly facing tines 114F, and the rearwardmost pair of sleeves 152 including rearwardly facing tines 114R. Having tines 114F and 114R project in different directions may help prevent migration of tissue stimulator 100 within tissue 138.

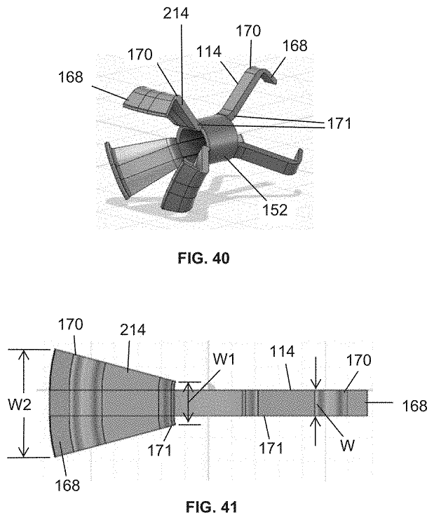

An alternative embodiment of a tine 214 is illustrated in FIGS. 40-41. As seen here and in the figures above, tines 114 may have a constant width W along their entire length. In other embodiments, as illustrated here, some tines 214 may be tapered such that they have a first width W1 at proximal end 171 of tine 214, and a second width W2 at an end of projection 168, with second width W2 being larger than first width W1. In such an embodiment, as seen in FIG. 40, such tapered tines 214 may provide an umbrella shape to the plurality of tines 214, which may increase the surface area of tines 214 as compared to tines 114 with a constant width W. This increased surface area may increase the retention force provided by tines 214, thereby helping decrease the chances of migration of tissue stimulator 100.

It is to be appreciated that in embodiments wherein tine 214 does not include a projection 168, second width W2 would be measured at distal end 170 of tine 214 itself.

As shown here, three tapered tines 214 are shown at one end of sleeve 152 and three tines 114 having a constant width are shown at the opposed end of sleeve 152. It is to be appreciated that tapered tines 214 may be connected to sleeve 152 without any tines 114 having a constant width. Additionally, it is to be appreciated that tines 214 with a tapered width can be mixed and matched with tines 114 having a constant width on a single sleeve 152, and that any number of tapered tines 214 and tines 114 of constant width may be implemented in tissue stimulator 100.

Several alternative embodiments and examples have been described and illustrated herein. A person of ordinary skill in the art would appreciate the features of the individual embodiments, and the possible combinations and variations of the components. A person of ordinary skill in the art would further appreciate that any of the embodiments could be provided in any combination with the other embodiments disclosed herein. It is understood that the invention may be embodied in other specific forms without departing from the spirit or central characteristics thereof. The present examples and embodiments, therefore, are to be considered in all respects as illustrative and not restrictive, and the invention is not to be limited to the details given herein. Terms “top,” “upper,” “bottom,” “lower,” “left,” “right,” and the like, as used herein, are intended for illustrative purposes only and do not limit the embodiments in any way. When used in description of a method or process, the term “providing” (or variations thereof) as used herein means generally making an article available for further actions, and does not imply that the entity “providing” the article manufactured, assembled, or otherwise produced the article. Nothing in this specification should be construed as requiring a specific three dimensional orientation of structures in order to fall within the scope of this invention, unless explicitly specified by the claims. Additionally, the term “plurality,” as used herein, indicates any number greater than one, either disjunctively or conjunctively, as necessary, up to an infinite number. Accordingly, while the specific embodiments have been illustrated and described, numerous modifications come to mind without significantly departing from the spirit of the invention and the scope of protection is only limited by the scope of the accompanying claims.

1. An implantable tissue stimulator assembly comprising:
a stimulator having a housing, a plurality of electrodes positioned along the housing, and a plurality of tines extending outwardly from the housing; and
a first introducer comprising:
a first barrel including a first base and a hollow first cylinder extending outwardly from the first base, the first cylinder having an open first end and an open second end and being configured to receive the stimulator for insertion of a distal end of the stimulator into tissue; and
a first stylet including a first handle and a first shaft extending outwardly from the first handle, the first stylet configured to be inserted into the first end and out of the second end of the first cylinder such that a distal end of the first stylet extends beyond the open second end of the first cylinder.

2. The stimulator assembly of claim 1, wherein the plurality of tines includes a first set of tines proximate a distal end of the housing and a second set of tines proximate a proximal end of the housing.

3. The stimulator assembly of claim 2, wherein the first set of tines includes tines extending toward the distal end of the housing and tines extending toward the proximal end of the housing.

4. The stimulator assembly of claim 2, wherein the second set of tines includes tines extending outwardly from the housing and toward the distal end of the housing and tines extending outwardly from the housing and toward the proximal end of the housing.

5. The stimulator assembly of claim 1, further comprising a plurality of sleeves encircling the housing, a set of tines extending outwardly from each of the sleeves.

6. The stimulator assembly of claim 5, wherein three tines extend outwardly from each of the sleeves.

7. The stimulator assembly of claim 5, wherein a first pair of sleeves is positioned on the housing proximate a distal end of the housing, and a second pair of sleeves is positioned on the housing proximate a proximal end of the housing.

8. The stimulator assembly of claim 5, further comprising a channel formed on an interior surface each sleeve, and a tine base formed at a proximal end of each tine, each tine base being received in one of the channels.

9. The stimulator assembly of claim 1, further comprising a second introducer comprising:
a second barrel including a second base and a hollow second cylinder extending outwardly from the second base, the second cylinder configured to receive the stimulator for insertion of a proximal end of the stimulator into tissue; and
a second stylet including a second handle and a second shaft extending outwardly from the second handle, the second stylet being received in the second cylinder.

10. The stimulator assembly of claim 1, wherein the first stylet has a pointed tip.

11. The stimulator assembly of claim 1, wherein the first stylet has a curved tip.

12. The stimulator assembly of claim 1, wherein a tip of the first stylet extends beyond a distal end of the first barrel when the first stylet is received in the first cylinder.

13. The stimulator assembly of claim 1, wherein an open end of the first base is tapered inwardly toward an interior of the first base.

14. The stimulator assembly of claim 1, further comprising a pair of perforations extending and opposed from one another along the first base and the first cylinder.

15. The stimulator assembly of claim 1, further comprising an antenna positioned in the housing.

16. The stimulator assembly of claim 1, wherein at least one tine has one of a pointed tip and a rounded tip.

17. The stimulator assembly of claim 1, further comprising a lubricous material located on an exterior surface of at least one tine.

18. The stimulator assembly of claim 1, further comprising a projection extending from a distal end of each tine.

19. The stimulator assembly of claim 18, wherein the projections extend inwardly toward the housing of the tissue stimulator.

20. The stimulator assembly of claim 1, wherein at least one tine has a first width at a proximal end of the tine and a second width at a distal end of the tine, the second width being larger than the first width.

21. An implantable tissue stimulator assembly comprising:
a stimulator having a housing, a plurality of electrodes proximate a distal end of the housing, and a first plurality of tines extending outwardly from the housing proximate the distal end and a second plurality of tines extending outwardly from the housing proximate a proximal end of the housing;
a first introducer comprising:
a first barrel including a first base and a hollow first cylinder extending outwardly from the base, the first base and first cylinder including a pair of opposed perforations extending along a length of the first base and the first cylinder, the first cylinder having an open first end and an open second end and being configured to receive the distal end of the stimulator for insertion of the stimulator into tissue, the first tines being configured to be folded inwardly toward the housing when the stimulator is received in the first cylinder; and
a first stylet including a first handle and a first shaft extending outwardly from the first handle, the first stylet configured to be inserted into the first end and out of the second end of the first cylinder such that a distal end of the first stylet extends beyond the open second end of the first cylinder; and
a second introducer configured to receive the proximal end of the stimulator for insertion of the stimulator into tissue, the second introducer comprising:
a second barrel including a second base and a hollow second cylinder extending outwardly from the second base, the second base and second cylinder including a pair of opposed perforations extending along a length of the second base and the second cylinder; and
a second stylet including a second handle and a second shaft extending outwardly from the second handle, the second stylet being received in the second cylinder.

22. A method of implanting a tissue stimulator comprising the steps of:
inserting a first introducer through an insertion site of tissue such that a tip of the first introducer is proximate a target site within the tissue, the first introducer including a first barrel including a first base and a hollow first cylinder extending outwardly from the first base, the first cylinder having an open first end and an open second end, and a first stylet including a first handle and a first shaft extending outwardly from the first handle, the first stylet being inserted into the first end and out of the second end of the first cylinder such that a distal end of the first stylet extends beyond the open second end of the first cylinder;
removing the first stylet from the first cylinder;
inserting a distal end of a stimulator into the first cylinder, the stimulator having a housing, a plurality of electrodes proximate a distal end of the housing, and a plurality of first tines extending outwardly from the housing proximate the plurality of electrodes, the first tines being folded inwardly toward the housing when the stimulator is in the first cylinder; and
removing the first barrel from the tissue such that the first tines move outwardly away from the housing and engage the tissue.

23. The method of claim 22, wherein the first barrel includes a pair of opposed perforations extending along a length of the first barrel and the step of removing the first barrel includes splitting the first barrel along the perforations into two portions as the first barrel is removed from the tissue.

24. The method of claim 22, further comprising the steps of:
inserting a second introducer through the insertion site such that a tip of the second introducer is proximate a surface of the tissue, the second introducer including a second barrel including a second base and a hollow second cylinder extending outwardly from the second base, and a second stylet including a second handle and a second shaft extending outwardly from the second handle, the second stylet being received in the second cylinder;
removing the second stylet from the second cylinder;
inserting a proximal end of the stimulator into the second cylinder, the stimulator having a plurality of second tines extending outwardly from the housing proximate the proximal end, the second tines being folded inwardly toward the housing when the stimulator is in the second cylinder; and
removing the second barrel from the tissue such that the second tines move outwardly away from the housing and engage the tissue.

25. An implantable tissue stimulator assembly comprising:
a stimulator having a housing, an antenna configured to accept wirelessly transmitted energy, a plurality of electrodes positioned along the housing, a circuit board having components configured to create pass pulsatile electrical currents to the electrodes, and a plurality of tines extending outwardly from the housing; and
a first introducer comprising:
a first barrel including a first base and a hollow first cylinder extending outwardly from the first base, the first cylinder having an open first end and an open second end and being configured to receive the stimulator for insertion of a distal end of the stimulator into tissue; and
a first stylet including a first handle and a first shaft extending outwardly from the first handle, the first stylet configured to be inserted into the first end and out of the second end of the first cylinder such that a distal end of the first stylet extends beyond the open second end of the first cylinder.
